(19) United States
(12) Patent Application Publication
(10) Pub. No.: US 2016/0166529 A1
(43) Pub. Date: Jun. 16, 2016

(54) Formulations for Cathepsin K Inhibitors with Vitamin D

(71) Applicants: Majid MAHJOUR, West Point, PA (US); Decheng MA, Flourtown, PA (US); Christina Marie BACCI, West Point, PA (US); Julianne Margaret FARABAUGH, West Point, PA (US); Justin MOSER, West Point, PA (US); Lixia CAI, West Point, PA (US); Stephen L. CONWAY, West Point, PA (US)

(72) Inventors: Majid Mahjour, Schwenksville, PA (US); Decheng Ma, Flourtown, PA (US); Christina Marie Bacci, Lansdale, PA (US); Julianne Margaret Farabaugh, North Wales, PA (US); Justin Moser, Collegeville, PA (US); Lixia Cai, Maple Glen, PA (US); Stephen L. Conway, Jenkintown, PA (US)

(73) Assignee: Merck Sharp & Dohme Corp., Rahway, NJ (US)

(21) Appl. No.: 14/903,662

(22) PCT Filed: Jul. 7, 2014
(86) PCT No.: PCT/US14/45511

§ 371 Date: Jan. 8, 2016

Related U.S. Application Data
(60) Provisional application No. 61/845,124, filed on Jul. 11, 2013.

(51) Int. Cl. A61K 31/277; A61K 9/20; A61J 3/10; A61K 31/593

(52) U.S. Cl. CPC A61K 31/277; A61K 31/593; A61K 9/2095; A61K 9/2054; A61K 9/2013; A61J 3/10

(57) ABSTRACT

The instant invention relates to pharmaceutical compositions comprising cathespin K inhibitors and Vitamin D. Also disclosed are processes for making said pharmaceutical compositions,

### BACKGROUND OF THE INVENTION

This invention relates to formulations comprising cathepsin K inhibitors and Vitamin D.

A variety of cathepsin K inhibitors have been disclosed for the treatment of various disorders related to cathepsin K functioning, including osteoporosis, glucocorticoid induced osteoporosis, Paget's disease, abnormally increased bone turn over, tooth loss, bone fractures, rheumatoid arthritis, osteoarthritis, periprosthetic osteolysis, osteogenesis imperfecta, atherosclerosis, obesity, glaucoma, chronic obstructive pulmonary disease and cancer including metastatic bone disease, hypercalcemia of malignancy, and multiple myeloma. Representative examples of cathepsin K inhibitors include those disclosed in International Publication WO03/075836, which published on Sep. 18, 2003, to Merck & Co., Inc. & Axys Pharmaceuticals, which is hereby incorporated by reference in its entirety.

Cathepsin K inhibitors can be formulated for oral dosing as tablets, by using a direct compression, wet granulation or roller compaction method. Similarly, cathepsin K inhibitors can be formulated for oral dosing as gelatin capsules, as a liquid in a soft capsule, or dry powder or semi-solid in a hard capsule. In addition, cathepsin K inhibitors can be formulated for intravenous dosing.

Vitamin D is a group of fat-soluble secosteroids, the two major physiologically relevant forms of which are vitamin D₂ (ergocalciferol) and vitamin D₃ (cholecalciferol). Vitamin D without a subscript refers to either D₂ or D₃ or both. Vitamin D₃ (“VitD3”) is produced in the skin of vertebrates after exposure to ultraviolet B light from the sun or artificial sources, and occurs naturally in fish and a few other foods. One of the most important roles of vitamin D is to maintain skeletal calcium balance by promoting calcium absorption in the intestines, promoting bone resorption by increasing osteoclast number, maintaining calcium and phosphate levels for bone formation, and allowing proper functioning of parathyroid hormone to maintain serum calcium levels.

The pharmaceutical compositions of the instant invention include fixed dose combinations of cathepsin K inhibitors with Vitamin D.

### SUMMARY OF THE INVENTION

The instant invention relates to pharmaceutical compositions comprising cathespin K inhibitors and Vitamin D. Also disclosed are processes for making said pharmaceutical compositions.

### DETAILED DESCRIPTION OF THE INVENTION

The instant invention relates to pharmaceutical compositions comprising cathespin K inhibitors and Vitamin D.

A particularly effective cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide,

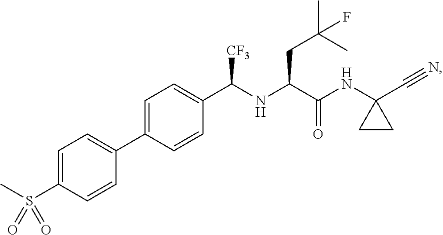 [SMILES: CC(C)(F)C[C@H](N[C@@H](c1ccc(-c2ccc(S(C)(=O)=O)cc2)cc1)C(F)(F)F)C(=O)NC1(C#N)CC1]

which can be prepared by procedures described in: International Publication WO03/075836, which published on Sep. 18, 2003, to Merck & Co., Inc. &. Axys Pharmaceuticals; International Publication WO2006/017455, which published on Feb. 16, 2006, to Merck & Co., Inc.; U.S. Publication US2006-0052642, which published on Mar. 09, 2006; U.S. Publication US2005-0234128, which published on Oct. 20, 2005, to Merck & Co., Inc.; all of which are hereby incorporated by reference in their entirety. This compound is also known by its generic name, odanacatib.

“Vitamin D” includes, but is not limited to, vitamin D₃ (cholecalciferol) and vitamin D₂ (ergocalciferol), which are naturally occurring, biologically inactive precursors of the hydroxylated biologically active metabolites of vitamin D: 1α-hydroxy vitamin D; 25-hydroxy vitamin D, and 1α,25-dihydroxy vitamin D. Vitamin D₂ and vitamin D₃ have the same biological efficacy in humans. When either vitamin D₂ or D₃ enters the circulation, it is hydroxylated by cytochrome P₄₅₀-vitamin D-25-hydroxylase to give 25-hydroxy vitamin D. The 25-hydroxy vitamin D metabolite is biologically inert and is further hydroxylated in the kidney by cytochrome P450-monooxygenase, 25 (OH) D-1α-hydroxylase to give 1,25-dihydroxy vitamin D. When serum calcium decreases, there is an increase in the production of parathyroid hormone (PTH), which regulates calcium homeostasis and increases plasma calcium levels by increasing the conversion of 25-hydroxy vitamin D to 1,25-dihydroxy vitamin D.

1,25-dihydroxy vitamin D is thought to be responsible for the effects of vitamin D on calcium and bone metabolism. The 1,25-dihydroxy metabolite is the active hormone required to maintain calcium absorption and skeletal integrity. Calcium homeostasis is maintained by 1,25 dihydroxy vitamin D by inducing monocytic stem cells to differentiate into osteoclasts and by maintaining calcium in the normal range, which results in bone mineralization by the deposition of calcium hydroxyapatite onto the bone surface, see Holick, M F, “Vitamin D photobiology, metabolism, and clinical applications”, In: DeGroot L, Besser H, Burger H G, et al., eds. Endocrinology, 3^{rd} ed., 990-1013 (1995). However, elevated levels of 1α,25-dihydroxy vitamin D₃ can result in an increase of calcium concentration in the blood and in the abnormal control of calcium concentration by bone metabolism, resulting in hypercalcemia. 1α,25-dihydroxy vitamin D₃ also indirectly regulates osteoclastic activity in bone metabolism and elevated levels may be expected to increase excessive bone resorption in osteoporosis.

In embodiments of the present invention, an appropriate amount of the vitamin D compound is chosen to provide adequate vitamin D nutrition during the dosing interval without interfering with the cathepsin K inhibitor's ability to obtain a bone resorption inhibiting effect. For oral compositions of the present invention comprising a cathepsin K inhibitor, and a vitamin D compound, an amount of the vitamin D compound comprises from about 100 IU (IU refers to International Units) to about 60,000 IU. Non-limiting examples of an oral amount of the vitamin D compound in embodiments of the present invention include, but are not limited to, dosages of 2,800 IU, 5,600 IU, 7,000 IU, 8,400 IU, 11,200 IU, 14,000 IU, 16,800 IU or 19,600 IU. Non-limiting examples of an oral amount of vitamin D for weekly dosing are 2,800 IU, 5,600 IU, 7,000 IU, 8,400 IU and 11,200 IU. Non-limiting examples of an oral amount of vitamin D for monthly dosing are 11,200 IU, 14,000 IU, 15,400 IU, 16,800 IU and 19,600 IU.

The invention contemplates the use of any pharmaceutically acceptable fillers/compression aids, disintegrants, super-disintegrants, lubricants, binders, surfactants, film coatings, and solvents. Examples of these components are set forth below and are described in more detail in the Handbook of Pharmaceutical Excipients, Second Edition, Ed. A. Wade and P. J. Weller, 1994, The Pharmaceutical Press, London, England.

The instant invention further comprises a pharmaceutical composition comprising by weight, about 10 mg to about 50 mg of a cathepsin K inhibitor, or a pharmaceutically acceptable salt thereof; about 0.14 mg to about 0.28 mg of Vitamin D, which is about 5400 IU to about 11,200 IU of Vitamin D; and from about 238 mg to 767 mg of excipients. In an embodiment of the pharmaceutical composition, the excipients comprise diluents, a binder, a disintegrant and a lubricant.

One (1) International Unit (IU) is equal to 0.025 μg, Vitamin D3. Thus, 5600 IU of Vitamin D3 is equal to 140 mcg (0.14 mg) of Vitamin D3 and 11200 IU of Vitamin D3 is equal to 280 mcg (0.28 mg) of Vitamin D3.

In an embodiment of the invention, the cathepsin K inhibitor is N¹-(1-cyanocyclopropyl)-4-fluoro-N-{(1S)-2,2,2-trifluoro-1-[4′-(methylsulfonyl)-1,1′-biphenyl-4-yl]ethyl}-L-leucinamide, or a pharmaceutically acceptable salt thereof. N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide is also known by its generic name, odanacatib.

In an embodiment of the invention, the pharmaceutical composition comprises by weight, 50 mg of a cathepsin K inhibitor, or a pharmaceutically acceptable salt thereof.

In an embodiment of the invention, the Vitamin D is Vitamin D3. In a class of the embodiment, the Vitamin D3 is provided as a stabilized formulation. A stabilized version of Vitamin D3 is manufactured by BASF.

In an embodiment of the invention, the diluents are selected from the group consisting of lactose anhydrous, lactose monohydrate, mannitol, microcrystalline cellulose, calcium phosphate and starch. In a class of the embodiment, the diluents are lactose monohydrate and microcrystalline cellulose.

Preferred brands of microcrystalline cellulose include Avicel® PH-101, Avicel® PH-102, Avicel® PH-105, and Avicel® Dry Granulation Excipient (DG).

In an embodiment of the invention, the binder is hydroxypropyl cellulose, polyvinylpyrrolidone or hydroxypropylmethylcellulose. In a class of the embodiment, the binder is hydroxypropyl cellulose.

In an embodiment of the invention the disintegrant is croscarmellose sodium, starch or sodium starch glycolate. In a class of the embodiment, the disintegrant is croscarmellose sodium.

In an embodiment of the invention, the lubricant is magnesium stearate or sodium stearyl fumarate. In a class of the embodiment, the lubricant is magnesium stearate.

The instant invention includes a process for the preparation of a tablet containing a cathepsin K inhibitor and Vitamin D, which process comprises:

(a) forming a powder blend of the cathepsin K inhibitor with excipients,

(b) granulating the powder blend to form granules,

(c) mixing the milled granules with Vitamin D granules and extragranular excipients,

(d) lubricating the mixture, and

(e) compressing the lubricated mixture into a tablet.

In an embodiment of the process, the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof.

In an embodiment of the process, combining the Vitamin D3 granules and extragranular excipients make the Vitamin D3 granules compressible. In an embodiment of the process, the extragranular excipients comprise a diluent and a disintegrant. In a class of the invention, the extragranular excipients comprise microcrystalline cellulose and croscarmellose sodium.

In an embodiment of the process, the excipients comprise diluents, a binder, and a disintegrant.

In an embodiment of the process, the diluents are selected from the group consisting of lactose anhydrous, lactose monohydrate, mannitol, microcrystalline cellulose, calcium phosphate and starch. In a class of the embodiment, the diluents are lactose monohydrate and microcrystalline cellulose.

In an embodiment of the process, the binder is hydroxypropyl cellulose, polyvinylpyrrolidone or hydroxypropylmethylcellulose. In a class of the embodiment, the binder is hydroxypropyl cellulose.

In an embodiment of the process, the disintegrant is croscarmellose sodium, starch or sodium starch glycolate. In a class of the embodiment, the disintegrant is croscarmellose sodium.

In an embodiment of the process, the lubricant is magnesium stearate or sodium stearyl fumarate. In a class of the embodiment, the lubricant is magnesium stearate.

The instant invention further includes a process for the preparation of a tablet containing a cathepsin K inhibitor and Vitamin D, which process comprises:

(a) forming a powder blend of the cathepsin K inhibitor with excipients,

(b) wet granulating the powder blend to form granules,

(c) drying the granules,

(d) milling the granules,

(e) mixing the milled granules with Vitamin D granules and extragranular excipients,

(f) lubricating the mixture, and

(g) compressing the lubricated mixture into a tablet.

In an embodiment of the process, the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof.

In an embodiment of the process, combining the Vitamin D3 granules and extragranular excipients make the Vitamin D3 granules compressible. In an embodiment of the process, the extragranular excipients comprise a diluent and a disintegrant. In a class of the invention, the extragranular excipients comprise microcrystalline cellulose and croscarmellose sodium.

In an embodiment of the process, the excipients comprise diluents, a binder, and a disintegrant.

In an embodiment of the process, the diluents are selected from the group consisting of lactose anhydrous, lactose monohydrate, mannitol, microcrystalline cellulose, calcium phosphate and starch. In a class of the embodiment, the diluents are lactose monohydrate and microcrystalline cellulose.

In an embodiment of the process, the binder is hydroxypropyl cellulose, polyvinylpyrrolidone or hydroxypropylmethylcellulose. In a class of the embodiment, the binder is hydroxypropyl cellulose.

In an embodiment of the process, the disintegrant is croscarmellose sodium, starch or sodium starch glycolate. In a class of the embodiment, the disintegrant is croscarmellose sodium.

In an embodiment of the process, the lubricant is magnesium stearate or sodium stearyl fumarate. In a class of the embodiment, the lubricant is magnesium stearate.

The pharmaceutical tablet compositions of the present invention may also contain one or more additional formulation ingredients that may be selected from a wide variety of excipients known in the pharmaceutical formulation art. According to the desired properties of the tablet, any number of ingredients may be selected, alone or in combination, based upon their known uses in preparing tablet compositions. Such ingredients include, but are not limited to, diluents, binders, compression aids, disintegrants, lubricants, flavors, flavor enhancers, sweeteners, preservatives, colorants and coatings.

The term “tablet” as used herein is intended to encompass compressed pharmaceutical dosage formulations of all shapes and sizes, whether uncoated or coated. Substances which may be used for coating include hydroxypropylmethylcellulose, hydroxypropylcellulose, titanium dioxide, talc, sweeteners and colorants.

The pharmaceutical compositions of the present invention are potentially useful in the therapeutic or prophylactic treatment of disorders including, but not limited to: osteoporosis, glucocorticoid induced osteoporosis, Paget's disease, abnormally increased bone turn over, tooth loss, bone fractures, rheumatoid arthritis, osteoarthritis, periprosthetic osteolysis, osteogenesis imperfecta, atherosclerosis, obesity, glaucoma, chronic obstructive pulmonary disease and cancer including metastatic bone disease, hypercalcemia of malignancy, and multiple myeloma.

In an embodiment of the invention, the cathepsin K inhibitor granulation consists of: 0.5 to 40% of a cathepsin K inhibitor or salt; 54% to 95.6% of a diluent or diluents; 0.5-2% of a lubricant. The cathepsin K inhibitor granulation can further, include 3-4% of a binder, as either a dry add or a binder solution. A class of the embodiment consists of 0.5 to 40% of N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide (odanacatib); 27% to 47.8% of lactose (as a diluent); 27% to 47.8% of microcrystalline cellulose (as a diluent); and 0.5-2% of magnesium stearate (as a lubricant).

In an embodiment of the invention, there is a 12.5% to 25% drug load of odanacatib in the granulation. In a class of the invention, there is a 12.5% drug load of odanacatib in the granulation. In another class of the invention, there is a 20% drug load of odanacatib in the granulation. In another class of the invention, there is a 25% drug load of odanacatib in the granulation.

The following examples are given for the purpose of illustrating the present invention and shall not be construed as being limitations on the scope of the invention.

As an example of the invention, the odanacatib granules, Vitamin D3 granules and extragranular excipients are combined as follows:

| | 50 mg Odanacatib | | | |
|---|---|---|---|---|
| Component | VitD3 5600 (IU) | | VitD3 11200 (IU) | |
| Drug Load (of | 12.5 | 25 | 12.5 | 25 |
| odanacatib in base | | | | |
| granulation) (%) | | | | |
| Amount of odanacatib | 400 | 200 | 400 | 200 |
| base granulation (mg) | | | | |
| VitD3 (mg) | 0.135 | 0.135 | 0.28 | 0.28 |
| Approximate weight of | 56 | 56 | 112 | 112 |
| Vitamin D3 granulation | | | | |
| (mg) | | | | |
| Additional | ~69-160 | ~144-259 | ~113-305 | ~88-305 |
| extragranular excipients | | | | |
| (mg) | | | | |
| Tablet Weight (mg) | 525-616 | 400-525 | 625-817 | 400-617 |

The odanacatib base granulation comprises odanacatib, diluents, a binder and a disintegrant. In an embodiment of the invention, the diluents are selected from the group consisting of lactose anhydrous, lactose monohydrate, mannitol, microcrystalline cellulose, calcium phosphate and starch. In a class of the embodiment, the diluents are lactose monohydrate and microcrystalline cellulose.

In an embodiment of the invention, the binder is hydroxypropyl cellulose, polyvinylpyrrolidone or hydroxypropylmethylcellulose. In a class of the embodiment, the binder is hydroxypropyl cellulose.

In an embodiment of the invention, the disintegrant is croscarmellose sodium, starch or sodium starch glycolate. In a class of the embodiment, the disintegrant is croscarmellose sodium.

As an example of the invention, the odanacatib granulation comprises odanacatib, hydroxypropyl cellulose, microcrystalline cellulose, lactose monohydrate, and croscarmellose sodium.

In an embodiment of the invention, the Vitamin D3 granulation comprises 100,000 IU/g, Gelatin coated, Pharmaceutical Grade Dry Vitamin D3. As used herein, the terms “Vitamin D3 granulation” and “Vitamin D3 granules” can be used interchangeably.

Specific examples of Vitamin D3 granulations are described as follows:

| | Supplier | | | | | | | | | |
|---|---|---|---|---|---|---|---|---|---|---|
| | | Zhejiang Garden | | | | | | | Ash- | |
| | BASF | (Huayuan Bio) | | Kingdomway | | NHU | WeiShi Bio | | Supreme | DSM |
| | Concentration | | | | | | | | | |
| Excipient | 100,000 | 100,000 | 500,000 | 100,000 | 500,000 | 100,000 | 100,000 | 200,000 | 100,000 | 100,000 |
| Cholecalciferol/VD3 crystal | 0.25-0.27 | 0.225%~0.275% | 0.01% | 0.20% | 1% | Y | 0.25-2.125% | | Y | Y |
| Medium Chain Triglycerides | 15.5-18.5 | | | | | | | | | Y |
| Starch sodium octenyl | | | | | | | 60% | 60% | | |
| succinate | | | | | | | | | | |
| Sodium ascorbate | | | | | | | | | | Y |
| Gelatin | 19.5-22.5 | | | | | | | | | |
| Gum arabic | | | | | | | 10% | 10% | | |
| Sucrose/Sugar | 29.0-32.0 | | | 5.00% | 5.00% | Y | 27.875-29.75% | | | Y |
| Modified Food Starch | 24-29 | | | 30.00% | 30.00% | Y | | | | Y |
| Butylated hydroxy toluene/ | 0.8-0.9 | | | 1.00% | 1.00% | | | | Y | |
| BHT | | | | | | | | | | |
| Sodium alummium silicate | 0.1-0.3 | | | | | | | | | |
| Vegetable wax | | | | | | | | | Y | |
| Butylated hydroxy anisole/ | | | | | | | | | Y | |
| BHA | | | | | | | | | | |
| Malto dextrin/β-Cyclodextrin | | 99.75% | 99.99% | 53.80% | 53.00% | Y | | | | |
| Silicon Dioxide | | | | | | | | | | Y |
| Tricalcium phosphate | | | | | | | | | | |
| dl-alpha-tocopherol | | | | | | | | | | Y |
| Vegetable/Plant oil | | | | 10.00% | 10.00% | Y | | | | |
| Water | 2.0-5.0 | | | | | Y | | | | |

In an embodiment of the invention, the extragranular excipients comprise a diluent and a disintegrant.

As an example of the invention, the extragranular excipients comprise microcrystalline cellulose and croscarmellose sodium.

As another example of the invention, the odanacatib granulation, Vitamin D3 granulation and extragranular excipients are combined as follows:

| | 10 mg Odanacatib | | | |
|---|---|---|---|---|
| Component | VitD3 5600 (IU) | | VitD3 11200 (IU) | |
| Drug Load (of | 12.5 | 25 | 12.5 | 25 |
| odanacatib in base | | | | |
| granulation) (%) | | | | |
| Amount of odanacatib | 80 | 40 | 80 | 40 |
| granulation (mg) | | | | |
| VitD3 (mg) | 0.135 | 0.135 | 0.28 | 0.28 |
| Approximate weight of | 56 | 56 | 112 | 112 |
| Vitamin D3 granulation | | | | |
| (mg) | | | | |
| Additional | ~112-168 | 112-168 | ~224-336 | ~224-336 |
| extragranular excipients | | | | |
| Tablet Weight (mg) | ~245-304 | ~209-265 | ~416-528 | ~376-488 |

The odanacatib granulation comprises odanacatib, diluents, a binder and a disintegrant. In an embodiment of the invention, the diluents are selected from the group consisting of lactose anhydrous, lactose monohydrate, mannitol, microcrystalline cellulose, calcium phosphate and starch. In a class of the embodiment, the diluents are lactose monohydrate and microcrystalline cellulose.

In an embodiment of the invention, the binder is hydroxypropyl cellulose, polyvinylpyrrolidone or hydroxypropylmethylcellulose. In a class of the embodiment, the binder is hydroxypropyl cellulose.

In an embodiment of the invention the disintegrant is croscarmellose sodium, starch or sodium starch glycolate. In a class of the embodiment, the disintegrant is croscarmellose sodium.

As an example of the invention, the odanacatib granulation comprises odanacatib, hydroxypropyl cellulose, microcrystalline cellulose, lactose monohydrate, and croscarmellose sodium.

In an embodiment of the invention, the Vitamin D3 granulation comprises 100,000 IU/g, Gelatin coated, Pharmaceutical Grade Dry Vitamin D3.

As an example of the invention, the extragranular excipients comprise a diluent and a disintegrant.

As an example of the invention, the extragranular excipients comprise microcrystalline cellulose and croscarmellose sodium.

### EXAMPLE 1

### Preparation of Tablets Containing 50 mg Odanacatib and 5600 IU Vitamin D3 Tablets (12.5% Drug Load of Odanacatib in Granulation)

Drug Product Composition

| Components | 5600 IU Vitamin D3 |
|---|---|
| Core Tablet: | mg/tablet |
| Odanacatib, milled | 50.0 |
| Hydroxypropyl Cellulose, [Klucel | 12.0 |
| EXF] | |
| Cellulose, Microcrystalline, [Avicel | 80.0 |
| PH-101] | |
| Lactose, Monohydrate | 234.0 |
| Croscarmellose sodium | 24.0 |
| Water, Purified ^{†} | 160.0 |
| Vitamin D3, dry granules, Gelatin- | 56.0 |
| coated, 100,000 IU ^{††} | |
| Avicel Dry Granulation Excipient | 33.30 |
| (DG) | |
| Croscarmellose sodium | 31.50 |
| Magnesium Stearate | 4.20 |
| Total Tablet Weight | 525 |

^{†} Removed during processing
^{††} Amount of Vitamin D3 granulation will be adjusted based on the Vitamin D3 assay of the granulation and total weight will be adjusted by adjusting the amount of the Avicel DG

Odanacatib, croscarmellose sodium, and a mixture of microcrystalline cellulose and lactose monohydrate are dry blended in a high shear mixer, and then a 3% (wt./wt.) hydroxypropyl cellulose solution is sprayed onto the mixing powders to effect granulation. The wet granulate is dried in a fluid bed dryer, and the dried granulate is then milled. The milled granules are mixed with the Vitamin D3 granules, extragranular Avicel Dry Granulation Excipient (DG) and croscarmellose sodium. The resulting mixture is then lubricated with magnesium stearate in a blender. The lubricated blend is compressed into tablets on a rotary tablet press.

### EXAMPLE 2

### Preparation of Tablets Containing 50 mg Odanacatib and 8400 IU Vitamin D3 Tablets (12.5% Drug Load of Odanacatib in Granulation)

Drug Product Composition

| Components | 8400 IU Vitamin D3 |
|---|---|
| Core Tablet: | mg/tablet |
| Odanacatib, milled | 50.0 |
| Hydroxypropyl Cellulose, [Klucel | 12.0 |
| EXF] | |
| Cellulose, Microcrystalline, [Avicel | 80.0 |
| PH-101] | |
| Lactose, Monohydrate | 234.0 |
| Croscarmellose sodium | 24.0 |
| Water, Purified ^{†} | 160.0 |
| Vitamin D3, dry granules, Gelatin- | 84.0 |
| coated, 100,000 IU ^{††} | |
| Avicel Dry Granulation Excipient | 51.9 |
| (DG) | |
| Croscarmellose sodium | 34.50 |
| Magnesium Stearate | 4.60 |
| Total Tablet Weight | 575 |

^{†} Removed during processing
^{††} Amount of Vitamin D3 granulation will be adjusted based on the Vitamin D3 assay of the granulation and total weight will be adjusted by adjusting the amount of the Avicel DG

Odanacatib, croscarmellose sodium, and a mixture of microcrystalline cellulose and lactose monohydrate are dry blended in a high shear mixer, and then a 3% (wt./wt.) hydroxypropyl cellulose solution is sprayed onto the mixing powders to effect granulation. The wet granulate is dried in a fluid bed dryer, and the dried granulate is then milled. The milled granules are mixed with the Vitamin D3 granules, extragranular Avicel 101 (microcrystalline Cellulose 101) and croscarmellose sodium. The resulting mixture is then lubricated with magnesium stearate in a blender. The lubricated blend is compressed into tablets on a rotary tablet press.

### EXAMPLE 3

### Preparation of Tablets Containing 50 mg Odanacatib and 11200 IU Vitamin D3 Tablets (12.5% Drug Load of Odanacatib in Granulation)

Drug Product Composition

| Components | 11200 IU Vitamin D3 |
|---|---|
| Core Tablet: | mg/tablet |
| Odanacatib, milled | 50.0 |
| Hydroxypropyl Cellulose, [Klucel | 12.0 |
| EXF] | |
| Cellulose, Microcrystalline, [Avicel | 80.0 |
| PH-101] | |
| Lactose, Monohydrate | 234.0 |
| Croscarmellose sodium | 24.0 |
| Water, Purified ^{†} | 160.0 |
| Vitamin D3, dry granules, Gelatin- | 112.0 |
| coated, 100,000 IU ^{††} | |
| Avicel Dry Granulation Excipient | 70.5 |
| (DG) | |
| Croscarmellose sodium | 37.5 |
| Magnesium Stearate | 5.0 |
| Total Tablet Weight | 625 |

^{†} Removed during processing
^{††} Amount of Vitamin D3 granulation will be adjusted based on the Vitamin D3 assay of the granulation and total weight will be adjusted by adjusting the amount of the Avicel DG

Odanacatib, croscarmellose sodium, and a mixture of microcrystalline cellulose and lactose monohydrate are dry blended in a high shear mixer, and then a 3% (wt./wt.) hydroxypropyl cellulose solution is sprayed onto the mixing powders to effect granulation. The wet granulate is dried in a fluid bed dryer, and the dried granulate is then milled. The milled granules are mixed with the Vitamin D3 granules, extragranular Avicel Dry Granulation Excipient (DG) and croscarmellose sodium. The resulting mixture is then lubricated with magnesium stearate in a blender. The lubricated blend is compressed into tablets on a rotary tablet press.

### EXAMPLE 4

### Preparation of Tablets Containing 50 mg Odanacatib and 5600 IU Vitamin D3 Tablets (25% Drug Load of Odanacatib in Granulation)

| Components | 5600 IV Vitamin D3 |
|---|---|
| Core Tablet: | mg/tablet |
| Odanacatib, milled | 50.0 |
| Hydroxypropyl Cellulose, [Klucel | 6.0 |
| EXF] | |
| Cellulose, Microcrystalline, [Avicel | 40.0 |
| PH-101] | |
| Lactose, Monohydrate | 92.0 |
| Croscarmellose sodium | 12.0 |
| Water, Purified ^{†} | 80.0 |
| Vitamin D3, dry granules, Gelatin- | 56.0 |
| coated, 100,000 IU ^{††} | |
| Avicel Dry Granulation Excipient | 116.8 |
| (DG) | |
| Croscarmellose sodium | 24.0 |
| Magnesium Stearate | 3.20 |
| Total Tablet Weight | 400 |

^{†} Removed during processing
^{††} Amount of Vitamin D3 granulation will be adjusted based on the Vitamin D3 assay of the granulation and total weight will be adjusted by adjusting the amount of the Avicel DG

Odanacatib, croscarmellose sodium, and a mixture of microcrystalline cellulose and lactose monohydrate are dry blended in a high shear mixer, and then a 3% (wt./wt.) hydroxypropyl cellulose solution is sprayed onto the mixing powders to effect granulation. The wet granulate is dried in a fluid bed dryer, and the dried granulate is then milled. The milled granules are mixed with the Vitamin D3 granules, extragranular Avicel Dry Granulation Excipient (DG) and croscarmellose sodium. The resulting mixture is then lubricated with magnesium stearate in a blender. The lubricated blend is compressed into tablets on a rotary tablet press.

### EXAMPLE 5

### Preparation of Tablets Containing 50 mg Odanacatib and 8400 IU Vitamin D3 Tablets (25% Drug Load of Odanacatib in Granulation)

| Components | 8400 IU Vitamin D3 |
|---|---|
| Core Tablet: | mg/tablet |
| Odanacatib, milled | 50.0 |
| Hydroxypropyl Cellulose, [Klucel | 6.0 |
| EXF] | |
| Cellulose, Microcrystalline, [Avicel | 40.0 |
| PH-101] | |
| Lactose, Monohydrate | 92.0 |
| Croscarmellose sodium | 12.0 |
| Water, Purified ^{†} | 80.0 |
| Vitamin D3, dry granules, Gelatin- | 84.0 |
| coated, 100,000 IU ^{††} | |
| Avicel Dry Granulation Excipient | 88.80 |
| (DG) | |
| Croscarmellose sodium | 24.0 |
| Magnesium Stearate | 3.20 |
| Total Tablet Weight | 400 |

^{†} Removed during processing
^{††} Amount of Vitamin D3 granulation will be adjusted based on the Vitamin D3 assay of the granulation and total weight will be adjusted by adjusting the amount of the Avicel DG

Odanacatib, croscarmellose sodium, and a mixture of microcrystalline cellulose and lactose monohydrate are dry blended in a high shear mixer, and then a 3% (wt./wt.) hydroxypropyl cellulose solution is sprayed onto the mixing powders to effect granulation. The wet granulate is dried in a fluid bed dryer, and the dried granulate is then milled. The milled granules are mixed with the Vitamin D3 granules, extragranular Avicel Dry Granulation Excipient (DG) and croscarmellose sodium. The resulting mixture is then lubricated with magnesium stearate in a blender. The lubricated blend is compressed into tablets on a rotary tablet press.

### EXAMPLE 6

### Preparation of Tablets Containing 50 mg Odanacatib and 11200 IU Vitamin D3 Tablets (25% Drug Load of Odanacatib in Granulation)

| Components | 11200 IU Vitamin D3 |
|---|---|
| Core Tablet: | mg/tablet |
| Odanacatib, milled | 50.0 |
| Hydroxypropyl Cellulose, [Klucel | 6.0 |
| EXF] | |
| Cellulose, Microcrystalline, [Avicel | 40.0 |
| PH-101] | |
| Lactose, Monohydrate: Impalpable | 92.0 |
| [312] | |
| Croscarmellose sodium | 12.0 |
| Water, Purified ^{†} | 80.0 |
| Vitamin D3, dry granules, Gelatin- | 112.0 |
| coated, 100,000 IU [Pharm Grade]^{††} | |
| Avicel Dry Granulation Excipient | 60.80 |
| (DG) | |
| Croscarmellose sodium, Compendial | 24.0 |
| Magnesium Stearate | 3.20 |
| Total Tablet Weight | 400 |

^{†} Removed during processing
^{††}Amount of Vitamin D3 granulation will be adjusted based on the Vitamin D3 assay of the granulation and total weight will be adjusted by adjusting the amount of the Avicel DG

Odanacatib, croscarmellose sodium, and a mixture of microcrystalline cellulose and lactose monohydrate are dry blended in a high shear mixer, and then a 3% (wt./wt.) hydroxypropyl cellulose solution is sprayed onto the mixing powders to effect granulation. The wet granulate is dried in a fluid bed dryer, and the dried granulate is then milled. The milled granules are mixed with the Vitamin D3 granules, extragranular Avicel Dry Granulation Excipient (DG) and croscarmellose sodium. The resulting mixture is then lubricated with magnesium stearate in a blender. The lubricated blend is compressed into tablets on a rotary tablet press.

### EXAMPLE 7

### Preparation of Capsules Containing 50 mg Odanacatib and 5600 IU Vitamin D3 Tablets (25% Drug Load of Odanacatib in Granulation)

| Components | 5600 IU Vitamin D3 |
|---|---|
| CAPSULE: | mg/capsule |
| Odanacatib, milled | 50.0 |
| Hydroxypropyl Cellulose, [Klucel | 6.0 |
| EXF] | |
| Cellulose, Microcrystalline, | 40.0 |
| [Avicel PH-101] | |
| Lactose, Monohydrate | 92.0 |
| Croscarmellose sodium | 12.0 |
| Water, Purified ^{†} | 80.0 |
| Vitamin D3, dry granules, | 56.0 |
| Gelatin-coated, 100,000 IU [Pharm | |
| Grade]^{††} | |
| Avicel Dry Granulation Excipient | 60-116.8 |
| (DG) | |
| Croscarmellose sodium | 12-24.0 |
| Magnesium Stearate | 0.5-3.20 |
| Total Blend Weight | ≦400 |
| Coni-Snap Capsule Hard Gelatin | 1, 0, or 00 |

^{†} Removed during processing
^{††}Amount of Vitamin D3 granulation will be adjusted based on the Vitamin D3 assay of the granulation and total weight will be adjusted by adjusting the amount of the Avicel DG

Odanacatib, croscarmellose sodium, and a mixture of microcrystalline cellulose and lactose monohydrate are dry blended in a high shear mixer, and then a 3% (wt./wt.) hydroxypropyl cellulose solution is sprayed onto the mixing powders to effect granulation. The wet granulate is dried in a fluid bed dryer, and the dried granulate is then milled. The milled granules are mixed with the Vitamin D3 granules, extragranular Avicel Dry Granulation Excipient (DG) and croscarmellose sodium. The resulting mixture is then lubricated with magnesium stearate in a blender. The lubricated blend is filled in capsules using a suitable encapsulation machine.

### EXAMPLE 8

### Preparation Of Tablets Containing 50 mg Odanacatib and 5600 IU Vitamin D3 Tablets (20% Drug Load of Odanacatib in Granulation)

| Components | 5600 IU Vitamin D3 |
|---|---|
| Core Tablet: | mg/tablet |
| Odanacatib | 50.0 |
| Hydroxypropyl Cellulose (Klucel EXF) | 7.50 |
| Microcrystalline Cellulose (Avicel PH-101) | 50.0 |
| Lactose Monohydrate: Impalpable (312) | 127.5 |
| Croscarmellose Sodium | 15.0 |
| Purified Water ^{‡} | |
| Vitamin D3, dry granules, Gelatin-coated, | 56.0 |
| 100,000 IU [Pharm Grade]^{§} | |
| Avicel PH-101 | 170.0 |
| Croscarmellose Sodium | 20.0 |
| Magnesium Stearate | 4.0 |
| Total Tablet Weight | 500 |

^{†} Compendial testing includes conformance to USP, NF, Ph. Eur., and/or JP
^{‡} Removed during processing
^{§}Amount of Vitamin D3 granulation will be adjusted based on the Vitamin D3 assay of the granulation and total weight will be adjusted by adjusting the amount of the Avicel PH101

Odanacatib, croscarmellose sodium, and a mixture of microcrystalline cellulose and lactose monohydrate are dry blended in a high shear mixer, and then a 3% (wt./wt.) hydroxypropyl cellulose solution is sprayed onto the mixing powders to effect granulation. The wet granulate is dried in a fluid bed dryer, and the dried granulate is then milled. The milled granules are mixed with the Vitamin D3 granules, extragranular Avicel Dry Granulation Excipient (DG) and croscarmellose sodium. The resulting mixture is then lubricated with magnesium stearate in a blender. The lubricated blend is compressed into tablets on a rotary tablet press.

### EXAMPLE 9

### Preparation of Tablets Containing 10 mg Odanacatib and 5600 IU Vitamin D3 Tablets (12.5% Drug Load of Odanacatib Ingranulation)

### Preparation of Tablets Containing 10 mg Odanacatib and 5600 IU Vitamin D3 Tablets (25% Drug Load of Odanacatib in Granulation)

### Preparation of Tablets Containing 10 mg Odanacatib and 11200 IU Vitamin D3 Tablets (12.5% Drug Load of Odanacatib in Granulation)

### Preparation of Tablets Containing 10 mg Odanacatib and 11200 IU Vitamin D3 Tablets (25% Drug Load of Odanacatib in Granulation)

| | 10 mg Odanacatib | | | |
|---|---|---|---|---|
| Component | VitD3 5600 IU | | VitD3 11200 IU | |
| Drug Load (in base | 12.5 | 25 | 12.5 | 25 |
| granulation) (%) | | | | |
| Amount of odanacatib | 80 | 40 | 80 | 40 |
| base granulation (mg) | | | | |
| VitD3 (mg) | 0.135 | 0.135 | 0.28 | 0.28 |
| Vitamin D3, dry | 56 | 56 | 112 | 112 |
| granules, Gelatin- | | | | |
| coated, 100,000 IU | | | | |
| [Pharm Grade] (mg) | | | | |
| Additional | ~112-168 | 112-168 | ~224-336 | ~224-336 |
| extragranular excipients | | | | |
| (mg) | | | | |
| Tablet Weight (mg) | ~245-304 | ~209-265 | ~416-528 | ~376-488 |

^{†} Removed during processing
^{††} Amount of Vitamin D3 granulation will be adjusted based on the Vitamin D3 assay of the granulation and total weight will be adjusted by adjusting the amount of the Avicel DG

The milled odanacatib base granules are mixed with the Vitamin D3 granules, extragranular Avicel Dry Granulation Excipient (DG) and croscarmellose sodium. The resulting mixture is then lubricated with magnesium stearate in a blender. The lubricated blend is compressed into tablets on a rotary tablet press.

### EXAMPLE 10

### Tensile Strength (MPa) of Tablets with Varying Amounts of Microcrystalline Cellulose (Avicel DG)

Composition of Tablet Formulations A-C tablets (50 mg/1.1200 IU, 25% DL, 400-587 mg image) and D (50 mg/11200 IU, 12.5% DL, 635 mg image)

| | | | | Odanacatib and |
|---|---|---|---|---|
| | | | | Vitamin D3 |
| | Odanacatib and Vitamin D3 | | | (50 mg/11200 |
| | (50 mg/11200 IU)- 25% DL\hat{ } | | | IU)- 12.5% DL |
| | A | B | C | D |
| Composition (%) | 400 mg | 450 mg | 587 mg | 625 mg |
| Odanacatib, 25% DL, | 50.00 | 44.44 | 34.07 | — |
| 6% Croscarmellose | | | | |
| Sodium | | | | |
| Odanacatib, 12.5% DL, | — | — | — | 64.00 |
| 6% Croscarmellose | | | | |
| Sodium | | | | |
| Vitamin D3, dry | 26.58 | 23.62 | 18.11 | 17.01 |
| granules, Gelatin- | | | | |
| coated, 100,000 IU | | | | |
| [Pharm Grade]* | | | | |
| Avicel DG | 16.63 | 25.13 | 40.87 | 12.19 |
| Croscarmellose Sodium | 6.00 | 6.00 | 6.13 | 6.00 |
| Magnesium Stearate | 0.80 | 0.80 | 0.82 | 0.80 |
| Total | 100.00 | 100.00 | 100.00 | 100.00 |
| Tablet Weight (mg) | 400.00 | 450.00 | 587.00 | 625.00 |

\hat{ }DL refers to Drug Load
*The actual potency for this lot is 105,340 IU/g.

Effect of Increasing Avicel DG Level on Odanacatib and Vitamin D3 Tablet (50 mg/11200 IU, 25% DL; 400-587 mg image) Tensile Strength (MPa)

| | Odanacatib and Vitamin D3 (50 mg/11200 IU)- | | | |
|---|---|---|---|---|
| | 25% DL | | | 12.5% DL |
| | A | B | C | D |
| CP (MPa)* | 400 mg | 450 mg | 587 mg | 625 mg |
| 100 | 1.0 | 1.3 | 1.6 | 1.0 |
| 200 | 1.9 | 2.5 | 3.2 | 2.1 |
| 300 | 2.4 | 3.1 | 4.2 | 2.7 |

*CP is Compaction Pressure, which is measured in Mega Pascals (MPa).

Increasing the Avicel DG level in the formulation improved the tensile strength of the tablets, showing from 1.9 MPa for A to 2.5 and 3.2 MPa for B and C, respectively, at 200 MPa pressure. To achieve a 2 MPa tensile strength for the formulation (D) containing 12.5% DL granulation, an image weight of 625 mg was necessary.

### EXAMPLE 11

### Tensile Strength (MPa) of Tablets with Varying Types of Microcrystalline Cellulose

Composition of Odanacatib and Vitamin D3 Tablet (50 mg/11200 IU, 587 mg image, 25% DL) with various types of Avicel

| | Odanacatib and Vitamin | | | | |
|---|---|---|---|---|---|
| | D3 (50 mg/11200 IU)- 25% DL | | | | |
| | | | | G | |
| | C | E | F | 587 mg | H |
| | 587 mg | 587 mg | 587 mg | Avicel | 587 mg |
| | Avicel | Avicel | Avicel | 101 + | Avicel |
| Composition (%) | DG | 101 | 102 | A-Tab | 105 |
| Odanacatib, 25% DL, | 34.07 | 34.07 | 34.07 | 34.07 | 34.07 |
| 6% Croscarmellose | | | | | |
| Sodium | | | | | |
| Vitamin D3, dry | 18.11 | 18.11 | 18.11 | 18.11 | 18.11 |
| granules, Gelatin- | | | | | |
| coated, 100,000 | | | | | |
| IU [Pharm Grade]* | | | | | |
| Avicel | 40.87 | 40.87 | 40.87 | 40.87 | 40.87 |
| Croscarmellose Sodium | 6.13 | 6.13 | 6.13 | 6.13 | 6.13 |
| Magnesium Stearate | 0.82 | 0.82 | 0.82 | 0.82 | 0.82 |
| Total | 100.00 | 100.00 | 100.00 | 100.00 | 100.00 |
| Tablet Weight (mg) | 587.00 | 587.00 | 587.00 | 587.00 | 587.00 |

*The actual potency for this lot is 105,340 IU/g.

Effect of Microcrystalline Cellulose type (Avicel) on Odanacatib and Vitamin D3 Tablet (50 mg/11200 IU; 587 mg image, 25% DL) Tensile Strength (MPa)

| | Odanacatib and Vitamin D3 | | | | |
|---|---|---|---|---|---|
| | (50 mg/11200 IU)- 25% DL granulation | | | | |
| | | | G | | |
| | E | F | 587 mg | H | C |
| | 587 mg | 587 mg | PH101 + | 587 mg | 587 mg |
| CP (MPa) | PH101 | PH102 | A-Tab | PH105 | DG 25% DL |
| 100 | 1.6 | 1.6 | 1.6 | 2.1 | 1.6 |
| 200 | 2.8 | 2.8 | 3.2 | 3.8 | 3.2 |
| 300 | 3.4 | 3.5 | 4.0 | 4.8 | 4.2 |

The formulation containing Avicel DG (C) had a tensile strength of 3.2 MPa, which was similar to the formulation with Avicel 101+A-Tab (G) and higher than the formulations with Avicel 101 (2.8 MPa) or Avicel 102 (2.8 MPa). The formulation with Avicel 105 had the highest tensile strength.

### EXAMPLE 12

### Tensile Strength (MPa) of Tablets with Varying Types of Microcrystalline Cellulose

Composition of Odanacatib and Vitamin D3 Tablet (50 mg/5600 IU, 25% DL, 400 mg image) with different Avicel type

| | Odanacatib and Vitamin D3 | | |
|---|---|---|---|
| | (50 mg/5600 IU)- 25% DL | | |
| | I | J | K |
| | 400 mg | 400 mg | 400 mg |
| | Avicel | Avicel | Avicel |
| Composition (%) | 105 | 101 | DG |
| Odanacatib, 25% DL, | 50 | 50 | 50.00 |
| 6% Croscarmellose Sodium | | | |
| Vitamin D3, dry granules, Gelatin- | 12.95 | 12.95 | 12.95 |
| coated, 100,000 IU [Pharm Grade]* | | | |
| Avicel | 30.25 | 30.25 | 30.25 |
| Croscarmellose Sodium | 6 | 6 | 6.00 |
| Magnesium Stearate | 0.8 | 0.8 | 0.80 |
| Total | 100 | 100 | 100.00 |
| Tablet Weight (mg) | 400 | 400 | 400 |

*The actual potency for this lot is 105,340 IU/g.

Effect of Avicel Type on Odanacatib and Vitamin D3 Tablet (50 mg/5600 U; 400 mg image, 25% DL) Tensile Strength (M-Pa)

| | 25% DL | | |
|---|---|---|---|
| CP | I | J | K |
| (MPa) | PH105 | PH101 | Avicel DG |
| 100 | 1.6 | 1.5 | 1.6 |
| 200 | 3.1 | 2.7 | 2.9 |
| 300 | 3.9 | 3.4 | 3.3 |

Similar to the H tablets, formulation with Avicel 105 had the best tensile strength of 3.1 MPa at 200 MPa pressure. Formulations with Avicel DG (2.9 MN had higher tensile strength than those with Avicel 101 (2.7 MPa).

### EXAMPLE 13

### Tensile Strength (MPa) of Tablets with Varying Types of Microcrystalline Cellulose

Composition of Odanacatib and Vitamin D3 (50 mg, 8400 IU, 12.5% DL) Formulations

| | Odanacatib and Vitamin D3 | | |
|---|---|---|---|
| | (50 mg/8400 IU, 12.5% DL) | | |
| | L | M | N |
| | 525 mg | 600 mg | 625 mg |
| | Avicel DG | Avicel DG | Avicel 101 |
| Composition (%) | Percentage | Percentage | Percentage |
| Odanacatib, 12.5% DL, | 69.57 | 66.67 | 64.00 |
| 6% Croscarmellose Sodium | | | |
| Vitamin D3, dry granules, | 13.51 | 13.29 | 12.76 |
| Gelatin-coated, 100,000 | | | |
| IU [Pharm Grade]* | | | |
| Avicel | 10.12 | 13.24 | 16.44 |
| Croscarmellose Sodium | 6.00 | 6.00 | 6.00 |
| Magnesium Stearate | 0.80 | 0.80 | 0.80 |
| Total (Percent) | 100.00 | 100.00 | 100.00 |
| Tablet Weight (mg) | 525.00 | 600.00 | 625.00 |

*The actual potency for this lot is 105,340 IU/g.

Composition of Odanacatib and Vitamin D3 (50 mg, 8400 IU, 25% DL) Formulations

| | Odanacatib and Vitamin D3 | |
|---|---|---|
| | (50 mg/8400 IU, 25% DL) | |
| | N | O |
| | 400 mg | 400 mg |
| | Avicel DG | Avicel 101 |
| Composition | Percentage | Percentage |
| Odanacatib and Vitamin D3, | 50.00 | 50.00 |
| 25% DL, 6% Croscarmellose Sodium | | |
| Vitamin D3, dry granules, Gelatin- | 19.94 | 19.94 |
| coated, 100,000 IU [Pharm Grade]* | | |
| Avicel | 23.27 | 23.27 |
| Croscarmellose Sodium | 6.00 | 6.00 |
| Magnesium Stearate | 0.80 | 0.80 |
| Total (Percent) | 100.00 | 100.00 |
| Tablet Weight (mg) | 400.00 | 400.00 |

*The actual potency for this lot is 105,340 IU/g.

Tablet strength (MPa) of Odanacatib and Vitamin D3 50 mg/8400 IU final blends compressed on the HB compaction simulator (at 120 mm/s)

| | | | | Odanacatib and | |
|---|---|---|---|---|---|
| | | | | Vitamin D3 | |
| | Odanacatib and Vitamin D3 | | | (50 mg/8400 IU, 25% | |
| | (50 mg/8400 IU, 12.5% DL | | | DL) | |
| CP | L | M | N | O | P |
| (MPa) | 525 mg | 600 mg | 625 mg | 400 mg | 400 mg |
| 100 | 1.1 | 1.2 | 1.5 | 1.2 | 1.4 |
| 200 | 2.3 | 2.4 | 2.8 | 2.4 | 2.6 |
| 300 | 2.9 | 2.9 | 3.4 | 3.0 | 3.3 |

The 12.5% DL formulations with Avicel DG, L (525 mg) and NI (600 mg) had almost similar tensile strengths of 2.3 and 2.4 MPa, respectively. The tensile strength of the 12.5% DL formulation with Avicel 101 (N, 625 mg) had a tensile strength of 2.8 MPa. The 25% DI, formulations with Avicel DG (O) and with Avicel 101 (P) at the 400 mg image had a tensile strength of 2.4 and 2.6 MPa, respectively.

What is claimed is:

1. A pharmaceutical composition comprising by weight, about 10 to 50 mg of a cathepsin K inhibitor, or a pharmaceutically acceptable salt thereof, about 0.14 to 0.28 mg of Vitamin D, and from about 238 to 767 mg of excipients selected from diluents, a binder, a lubricant, and a disintegrant.

2. The pharmaceutical composition of claim 1 wherein the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof.

3. The pharmaceutical composition of claim 1 comprising by weight, about 10 to 50 mg of a is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide about 0.14 to 0.28 mg of Vitamin D, and from about 238 to 767 mg of excipients selected from diluents, a binder, a lubricant, and a disintegrant.

4. The pharmaceutical composition of claim 1 wherein the Vitamin D is Vitamin D3.

5. The pharmaceutical composition of claim 1 wherein
the diluents are selected from the group consisting of lactose anhydrous, lactose monohydrate, mannitol, microcrystalline cellulose, calcium phosphate and starch;
the binder is hydroxypropyl cellulose, polyvinylpyrrolidone or hydroxypropylmethylcellulose;
the lubricant is magnesium stearate or sodium stearyl fumarate; and
the disintegrant is croscarmellose sodium, starch or sodium starch glycolate.

6. The pharmaceutical composition of claim 5 wherein the diluents are lactose monohydrate and microcrystalline cellulose; the binder is hydroxypropyl cellulose; the lubricant is magnesium stearate; and the disintegrant is croscarmellose sodium.

7. The pharmaceutical composition of claim 6 wherein the microcrystalline cellulose is selected from the group consisting of Avicel® PH-101, Avicel® PH-102, Avicel® PH-105, and Avicel® Dry Granulation Excipient.

8. The pharmaceutical composition of claim 7 wherein the microcrystalline cellulose is Avicel® Dry Granulation Excipient.

9. A pharmaceutical composition comprising 50 mg of N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide; 7,5 mg of hydroxypropyl cellulose; 220 mg of microcrystalline cellulose; 127.5 mg of lactose monohydrate; 35 mg of croscarmellose sodium; 56 mg of Vitamin D3 granules; and 4 mg of magnesium stearate.

10. A pharmaceutical composition comprising 50 mg of N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide; 7.5 mg of hydroxypropyl cellulose; 220 mg of microcrystalline cellulose; 127.5 mg of lactose monohydrate; 35 mg of croscarmellose sodium; 5600 IU of Vitamin D3; and 4 mg of magnesium stearate.

11. A process for the preparation of a tablet containing a cathepsin K inhibitor and Vitamin D, which process comprises:
(a) forming a powder blend of the cathepsin K inhibitor with excipients,
(b) wet granulating the powder blend to form granules,
(c) drying the granules,
(d) milling the granules,
(e) mixing the milled granules with Vitamin D granules and extragranular excipients,
(f) lubricating the mixture, and
(g) compressing the lubricated mixture into a tablet.

12. The process of claim 11 wherein the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof.

13. The process of claim 11 wherein the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide.

14. The process of claim 11 wherein the Vitamin D granules are Vitamin D3 granules, and the extragranular excipients comprise a diluent and a disintegrant.

15. The process of claim 14 wherein the extrangranular excipients are microcrystalline cellulose and croscarmellose sodium.

16. The process of claim 11 wherein the excipients comprise diluents, a binder, and a disintegrant.
